(19) United States
(12) Patent Application Publication
(10) Pub. No.: US 2016/0213456 A1
(43) Pub. Date: Jul. 28, 2016

(54) SILK MEDICAL DEVICE

(71) Applicant: Allergan, Inc., Irvine, CA (US)

(72) Inventor: Enrico Mortarino, Hickory, NC (US)

(21) Appl. No.: 15/001,483

(22) Filed: Jan. 20, 2016

Related U.S. Application Data
(63) Continuation of application No. 13/715,872, filed on Dec. 14, 2012, now abandoned.
Application No. 13/715,872 is a continuation-in-part of application No. 13/306,325, filed on Nov. 29, 2011.
Application No. 13/306,325 is a continuation-in-part of application No. 13/186,151, filed on Jul. 19, 2011.
Application No. 13/186,151 is a continuation-in-part of application No. 13/156,283, filed on Jun. 8, 2011, now abandoned.
Application No. 13/156,283 is a continuation-in-part of application No. 12/680,404, filed on Sep. 19, 2011, now abandoned.
(60) Provisional application No. 61/122,520, filed on Dec. 15, 2008.

(51) Int. Cl. A61F 2/00; A61F 2/12

(52) U.S. Cl. CPC A61F 2/0063; A61F 2/12; A61F 2002/0068; A61F 2240/001

(57) ABSTRACT

An implantable knitted silk mesh for use in human soft tissue support and repair having a particular knit pattern that substantially prevents unraveling and preserves the stability of the mesh when cut, the knitted mesh including at least two yarns laid in a knit direction and engaging each other to define a plurality of nodes.

### CROSS REFERENCE

This patent application is a continuation of U.S. patent application Ser. No. 13/715,872, filed Dec. 14, 2012, which is a continuation in part of U.S. patent application Ser. No. 13/306,325, filed Nov. 29, 2011, which is a continuation in part of U.S. patent application Ser. No. 13/186,151, filed Jul. 19, 2011, which is a continuation in part of U.S. patent application Ser. No. 13/156,283, filed Jun. 8, 2011, which is a continuation in part of U.S. patent application Ser. No. 12/680,404, filed Sep. 19, 2011, which is a national stage entry of PCT patent application number PCT/US09/63717, filed Nov. 9, 2009, which claims priority to and the benefit of U.S. provisional patent application No. 61/122,520, filed Dec. 15, 2008, all of which applications are expressly incorporated by reference herein in their entireties.

### BACKGROUND

The present invention is a biodegradable (synonymously bioresorbable), biocompatible knitted silk matrix, mesh or scaffold (the “device”) and methods for making and using the device in surgical and cosmetic procedures where soft tissue (i.e. a gland, organ, muscle, skin, ligament, tendon, cartilage, blood vessel or mesentery) support (through the load bearing function of the device) is desired, such as for example in breast reconstruction, breast augmentation, abdominal surgery, gastro-intestinal surgery, hernia repair and facial surgery.

Soft tissue support surgical meshes and scaffolds are known and are usually made of a synthetic polymer such as Teflon®, polypropylene, polyglycolic acid, polyester, or polyglactin 910. Biomaterials such a tissue based or tissue derived material, for example an acellular dermal matrix (“ADM”) obtained from human and animal derived dermis have also been used but do not have the mechanical integrity of high load demand applications (e.g. ligaments, tendons, muscle) or the appropriate biological functionality because most biomaterials either degrade too rapidly (e.g., collagen, PLA, PGA, or related copolymers) or are non-degradable (e.g., polyesters, metal), and in either case functional autologous tissue ingrowth (important to assist transfer of a load bearing function from an implanted biomaterial as the biomaterial is bioresorbed by the body) occurs very little or fails to occur. In certain instances a biomaterial may misdirect tissue differentiation and development (e.g. spontaneous bone formation, tumors) because it lacks biocompatibility with surrounding cells and tissue. As well, a biomaterial that fails to degrade typically is associated with chronic inflammation and such a response is detrimental to (i.e. weakens) surrounding and adjacent tissue.

Silk is a natural (non-synthetic) protein made of high strength fibroin fibers with mechanical properties similar to or better than many of synthetic high performance fibers. Silk is also stable at physiological temperatures in a wide range of pH, and is insoluble in most aqueous and organic solvents. As a protein, unlike the case with most if not all synthetic polymers, the degradation products (e.g. peptides, amino acids) of silk are biocompatible. Silk is non-mammalian derived and carries far less bioburden than other comparable natural biomaterials (e.g. bovine or porcine derived collagen). Silk, as the term is generally known in the art, means a filamentous fiber product secreted by an organism such as a silkworm or spider. Silks can be made by certain insects such as for example Bombyx mori silkworms, and Nephilia clavipes spiders. There are many variants of natural silk. Fibroin is produced and secreted by a silkworm's two silk glands. As fibroin leaves the glands it is coated with sericin a glue-like substance. Spider silk s produced as a single filament lacking the immunogenic protein sericin. Use of both silkworm silk and spider silk (from a natural source or made recombinantly) is within the scope of the present invention.

Silkworm silk has been used in biomedical applications. The Bombyx mori species of silkworm produces a silk fiber (a “bave”) and uses the fiber to build its cocoon. The bave as produced include two fibroin filaments or broins which are surrounded with a coating of the gummy, antigenic protein sericin. Silk fibers harvested for making textiles, sutures and clothing are not sericin extracted or are sericin depleted or only to a minor extent and typically the silk remains at least 10% to 26% by weight sericin. Retaining the sericin coating protects the frail fibroin filaments from fraying during textile manufacture. Hence textile grade silk is generally made of sericin coated silk fibroin fibers. Medical grade silkworm silk is used as either as virgin silk suture, where the sericin has not been removed, or as a silk suture from which the sericin has been removed and replaced with a wax or silicone coating to provide a barrier between the silk fibroin and the body tissue and cells. Thus there is a need for a sericin extracted implantable, bioresorbable silk device that promotes ingrowth of cells.

### SUMMARY

A device according to the present fulfills these needs and solves the indicated problems. The device in one embodiment is a knitted mesh having at least two yarns laid in a knit direction and engaging each other to define a plurality of nodes, the at least two yarns including a first yarn and a second yarn extending between and forming loops about two nodes, the second yarn having a higher tension at the two nodes than the first yarn, the second yarn substantially preventing the first yarn from moving at the two nodes and substantially preventing the knitted mesh from unraveling at the nodes. The device is a surgical mesh made of silk that is knitted, multi-filament, and bioengineered. It is mechanically strong, biocompatible, and long-term bioresorbable. The sericin-extracted silkworm fibroin fibers of the device retain their native protein structure and have not been dissolved and/or reconstituted.

“Bioresorbed” means that none or fewer than 10% of the silk fibroin fibers of the device can be seen to the naked (no magnification aid) eye upon visual inspection of the site of implantation of the device or of a biopsy specimen therefrom, and/or that the device is not palpable (i.e. cannot be felt by a surgeon at a time after the surgery during which the device was implanted) upon tactile manipulation of the dermal location of the patient at which the device was implanted. Typically either or both of these bioresorbed determinants occur about 1 to about 2 years are in vivo implantation of the device.

“About” means plus or minus ten percent of the quantify, number, range or parameter so qualified.

The device of the present invention is a sterile surgical mesh or scaffold available in a variety of shapes and sizes ready for use in open surgical or in laparoscopic procedures. The device is flexible and well-suited for delivery through a laparoscopic trocar due to its strength, tear resistance, suture retention, and ability to be cut in any direction. The device can provide immediate physical and mechanical stabilization of a tissue defect through the strength and porous (scaffold-like) construction of the device. The device can be used as a transitory scaffold for soft tissue support and repair to reinforce deficiencies where weakness or voids exist that require the addition of material to obtain the desired surgical outcome.

The device can comprise filament twisted silk yarns. The silk is made of silk fibroin fibers. The silk fibroin fibers are preferably sericin depleted or sericin extracted silk fibroin fibers. The device has an open pore knit structure. Significantly, after implantation the device and ingrown native tissue can maintain at least about 90% of the time zero device strength of the device at one month or at three months or at six months in vivo after the implantation. The device can be implanted without regard to side orientation of the device and the combined thickness of the device and ingrowth of native tissue scaffold increases with time in vivo in the patient.

As used herein, “fibroin” includes silkworm fibroin (i.e. from Bombyx mori) and fibroin-like fibers obtained from spiders (i.e. from Nephila clavipes). Alternatively, silk protein suitable for use in the present invention can be obtained from a solution containing a genetically engineered silk, such as from bacteria, yeast, mammalian cells, transgenic animals or transgenic plants. See, for example, WO 97/08315 and U.S. Pat. No. 5,245,012.

The device is a knitted silk fabric intended for implantation in a human body. The word “knit” is synonymous with the word “knitted”, so that a knit silk fabric is the same as a knitted silk fabric. The device can be a warp knit or can be weft knit silk fabric. Preferably, the device according of the present invention is a biocompatible, warp knit, multi-filament silk fabric. A woven material or fabric is made by weaving, which is a process that does not use needles, and results in a fabric with different characteristics. In particular, a woven fabric is made by a non-needle process using multiple yarns that interlace each other at right angles to form a structure wherein one set of yarn is parallel to the direction of fabric formation. Woven fabrics are classified as to weave or structure according to the manner in which warp and weft cross each other. The three main types of weaves (woven fabrics) are plain, twill, and satin. Woven (weaved) silk fabric, woven textiles and woven fabrics are not within the scope of the present invention. Non-woven fabrics are also not within the scope of the present invention. Non-woven (also refer to as bonded) fabrics are formed by having multiple fibers cohered together chemically or physically, without use of needles.

Unlike the excluded woven and non-woven materials, a knitted fabric is generally softer and more supple because its thread is treated differently. Thus a knitted fabric is made by using needles (such as for example the needles of a single or double bed knit machine) to pull threads up through the preceding thread formed into a loop by the needle. Because a knitted fabric is made using needles the knitted fabric can have one or multiple yarn intermeshing (also referred as interloping). Preferably, the device is made of biodegradable silk and is a biocompatible, non-woven, knit, multi-filament silk fabric or mesh.

Embodiments according to aspects of the present invention provide a biocompatible surgical silk mesh device for use in soft or hard tissue repair. Examples of soft tissue repair include hernia repair, rotator cuff repair, cosmetic surgery, implementation of a bladder sling, or the like. Examples of hard tissue repair, such as bone repair, involve reconstructive plastic surgery, ortho trauma, or the like.

Advantageously, the open structure of the device allows tissue ingrowth as the silk forming the device is bioresorbed, at a rate permitting smooth transfer of mechanical properties to the new tissue from the device. Furthermore, the device has a knit pattern that substantially or entirely prevents unraveling, especially when the device is cut. The device have a stable knit pattern made by knitting silk yarn with variations of tension between at least two yarns laid in a knit direction. For example, a first yarn and a second yarn may be laid in a knit direction to form “nodes” for a mesh device. The knit direction for the at least two yarns, for example, may be vertical during warp knitting or horizontal during weft knitting. The nodes of a mesh device, also known as intermesh loops, refer to intersections in the mesh device where the two yarns form a loop around a knitting needle. In some embodiments, the first yarn is applied to include greater slack than the second yarn, so that, when a load is applied to the mesh device, the first yarn is under a lower tension than the second device. A load that places the at least two yarns under tension may result, for example, when the mesh device is sutured or if there is pulling on the mesh device. The slack in the first yarn causes the first yarn to be effectively larger in diameter than the second yarn, so that the first yarn experiences greater frictional contact with the second yarn at a node and cannot move, or is “locked,” relative to the second yarn. Accordingly, this particular knit design may be referred to as a “node-lock” design.

The device bioresorbs at a rate sufficient that allows tissue in-growth while transferring the load-bearing responsibility to the native tissue. An embodiment of the device can be made from Bombyx mori silkworm silk fibroin or from spider silk. The raw silk fibers have a natural globular protein coating known as sericin, which may have antigenic properties and must be depleted before implantation. Accordingly, the yarn is taken through a depletion process as described, for example, by Gregory H. Altman et al., “Silk matrix for tissue engineered anterior cruciate ligaments,” Biomaterials 23 (2002), pp. 4131-4141, the contents of which are incorporated herein by reference. As a result, the silk material used in the device embodiments contains substantially no (less than 5%) sericin.

A device and a preferred process for making the device (a knitted silk mesh) within the scope of the present invention can comprise one or more of the following process steps: knitting a first silk yarn in a first wale direction using the pattern 3/1-1/1-1/3-3/3; knitting a second silk yarn in a second wale direction using the pattern 1/1-1/3-3/3-3/1, and; knitted a third silk yarn in a course direction using the pattern 7/7-9/9-7/7-9/9-7/7-9/9/-1/1-1/1-3/3-1/1-3/3-1/1. In this process the first wale direction knit upon the first silk yarn is carried out on a first needle bed and the second wale direction knit upon the second silk yarn is carried out on a second needle bed. Additionally in this process each of the three silk yarns is made with 3 ends of Td (denier count) 20/22 raw silk twisted together in the S direction to form a ply with 20 tpi (turns per inch) and further combining three of the resulting ply with 10 tpi. Furthermore, in this process the resulting knitted silk mesh has a stitch density (pick count) of 34 picks per centimeter with regard to the total picks count for the technical front face and the technical back face of the mesh. The Td (titer-denier) can be used to measure the fineness of reeled silk. The direction of twist in a yarn is indicated by S and Z. A yarn has an S twist when held vertically if spirals or helices around its central axis in the direction of slope conform to the letter S. If the spirals or helices in the direction of slope conform to the central portion of the letter Z then it is designated a Z twist.

A preferred embodiment of the device (a knitted silk mesh) can have the characteristic of: a thickness between about 0.6 mm and about 1.0 mm; pores with an average diameter greater than about 10,000 um²; a density of from about 0.14 mg/mm³ to about 0.18 mg/mm³; a burst strength of from about 0.54 MPa to about 1.27 MPa; a stiffness of between about 30 N/mm to about 50 N/mm, and; at least about 50% of the mass of the device bioresorbs after about 100 days after implantation in a human patient.

### DRAWINGS

The present invention can be more fully understood from the detailed description and the accompanying drawings, which are not necessarily to scale, wherein:

FIG. 1A is a photograph of a pattern layout for a silk-based scaffold design in accordance with the present invention.

FIGS. 1B and 1C illustrate an example pattern layout for the scaffold design of FIG. 1A including all pattern and ground bars according to aspects of the present invention.

FIGS. 1D and 1E illustrate an example pattern layout for a double needle bed mesh or scaffold according to aspects of the present invention from FIG. 1B for ground bar #4.

FIGS. 1F and 1G illustrate an example pattern layout for a double needle bed mesh or scaffold according to aspects of the present invention from FIG. 1B for pattern bar #5.

FIGS. 1H and 1I illustrate an example pattern layout for a double needle bed mesh or scaffold according to aspects of the present invention from FIG. 1B for ground bar #7.

FIG. 1J illustrates an example pattern simulation for a double needle bed mesh demonstrated in FIG. 1B according to aspects of the present invention.

FIG. 1K shows the yarn feed rates used during the knit process used to make the most preferred embodiment of the device within the scope of the present invention.

FIG. 2 illustrates the twisting and multi-ply nature of a yarn comprised of silk fibroin bundles as used in an embodiment of the present invention.

FIGS. 3A and 3B show respectively scanning electromicrographs (“SEM”) of native silk fibers and sericin extracted silk fibers, the latter being used to make the device. The size bar at the top of each Figure measures 20 microns.

FIG. 4 is a photograph of an embodiment (a knitted silk fabric ready for implantation) of a device within the scope of the present invention (placed above a millimeter ruler).

FIG. 5A is photograph at 16× magnification of a portion of the FIG. 4 embodiment.

FIG. 5B is photograph of the FIG. 4 embodiment showing the ease with which it can be cut without the fabric unraveling or fraying.

### DESCRIPTION

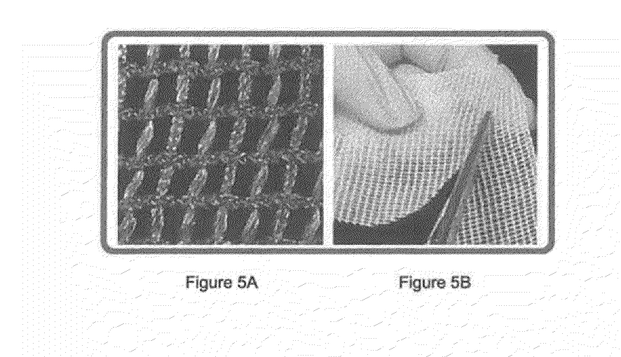

The present invention is based on discovery of an implantable, bioresorbable, biocompatible, knitted, porous silk mesh (the “device”) which upon implantation provides soft tissue support and, as the device bioresorbs, transfer of its load bearing (support) function to new tissue formed at the site of implantation. The device is preferably made from Bombyx mori silkworm silk. It can also be made from spider silk, including recombinantly made spider silk. The preferred knit pattern of the device accomplishes variation in tension between yarns at the knit nodes (the yarn interlocking loops) thereby preventing unraveling of the mesh when cut for use in surgery. FIG. 5A (left hand side) shows a 16× magnification of the device knit pattern and FIG. 5B (right hand side) shows ease of cutting without fraying or unraveling.

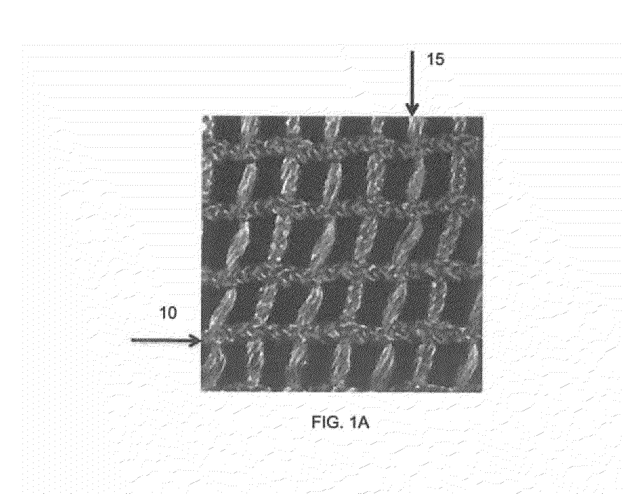

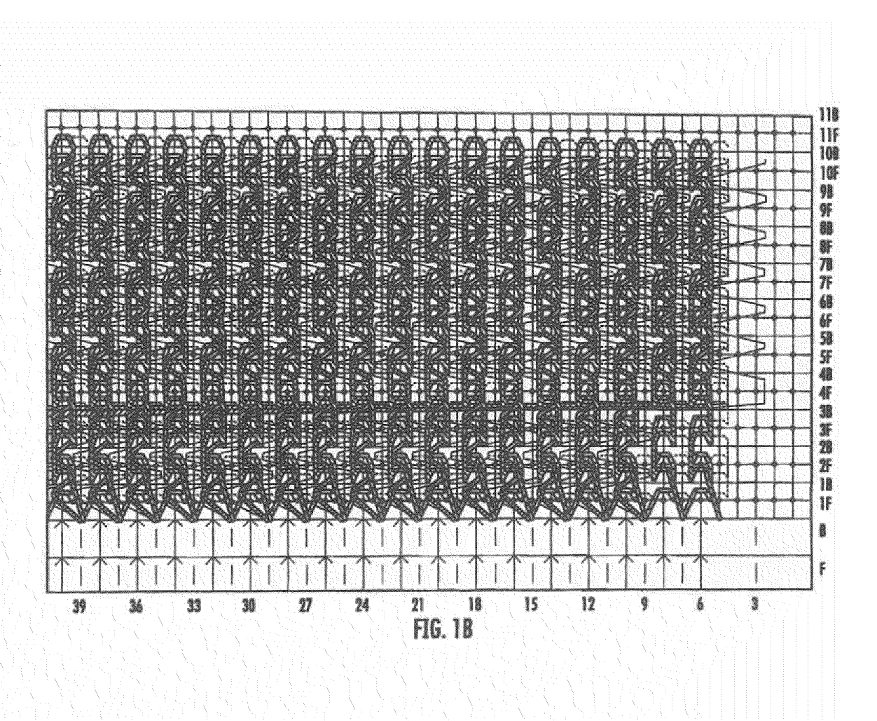

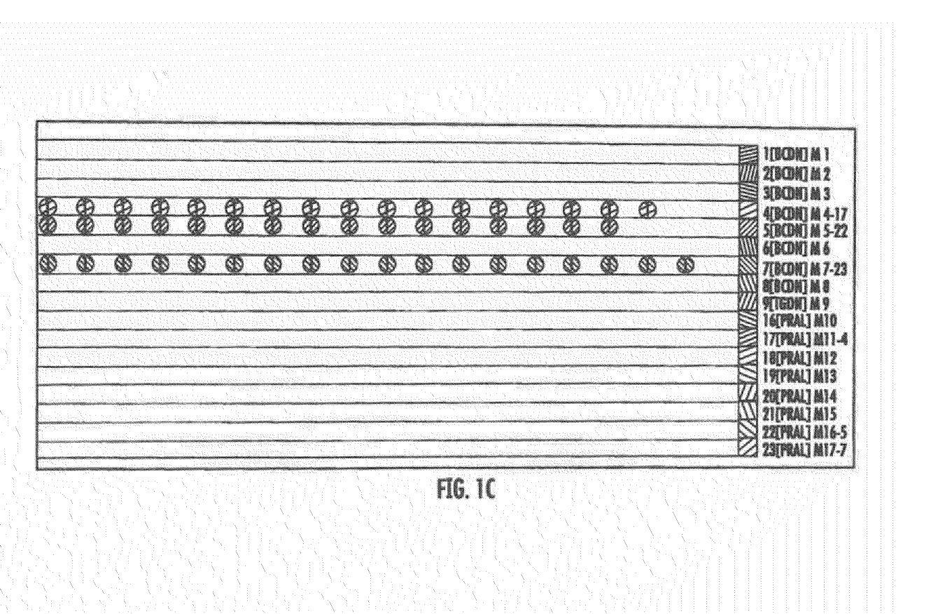

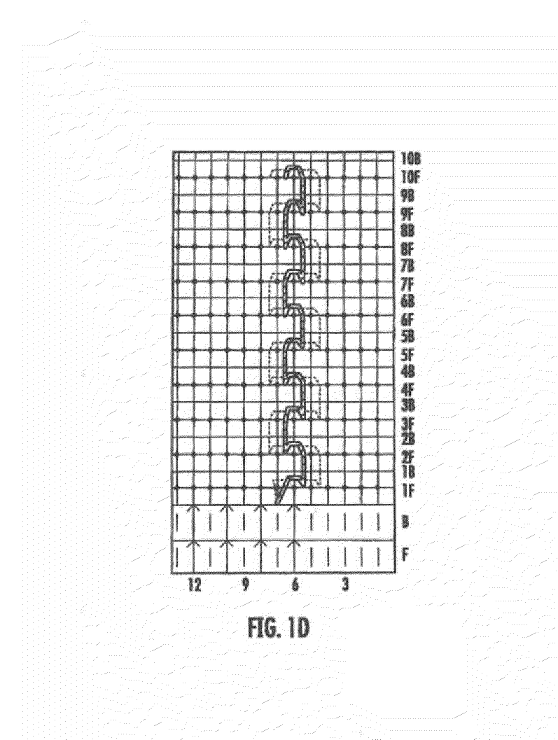

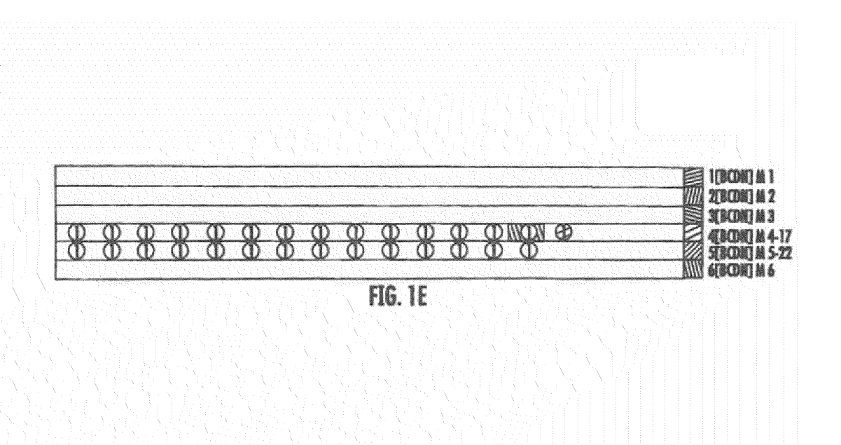

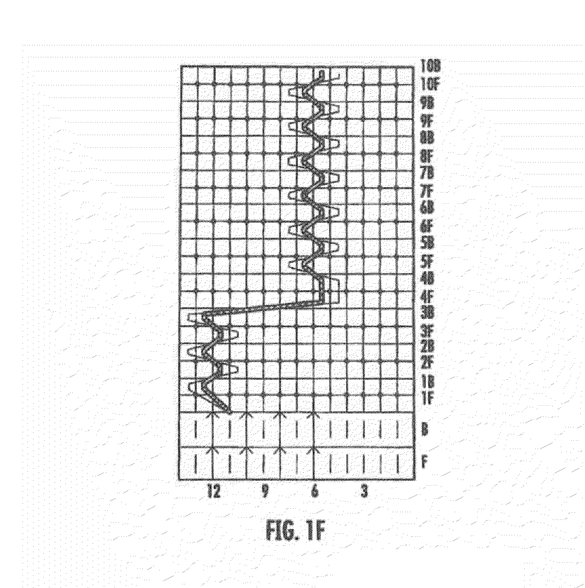

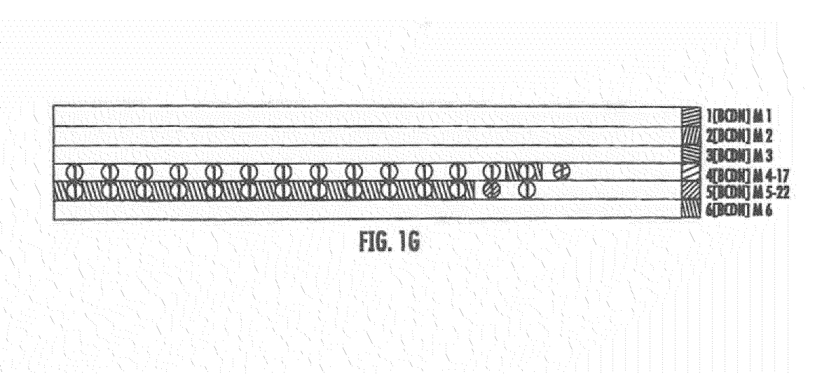

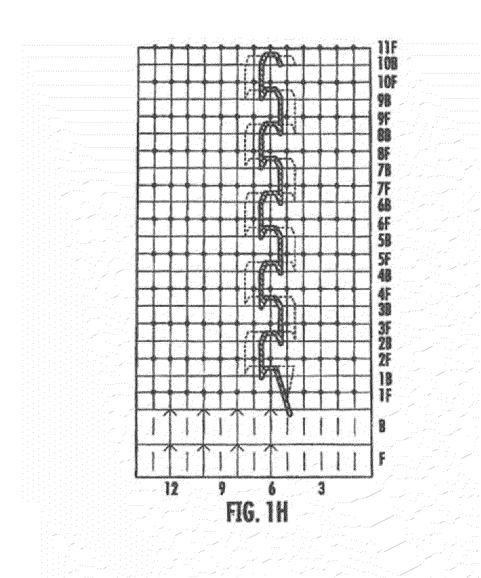

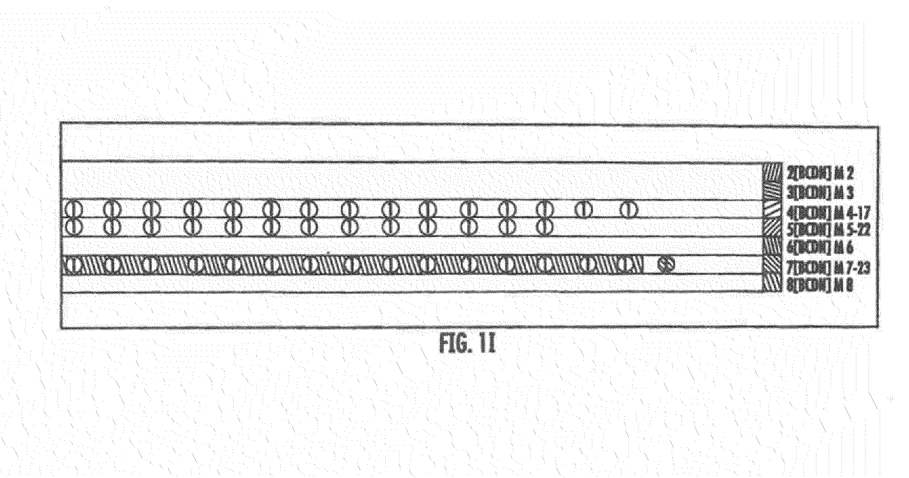

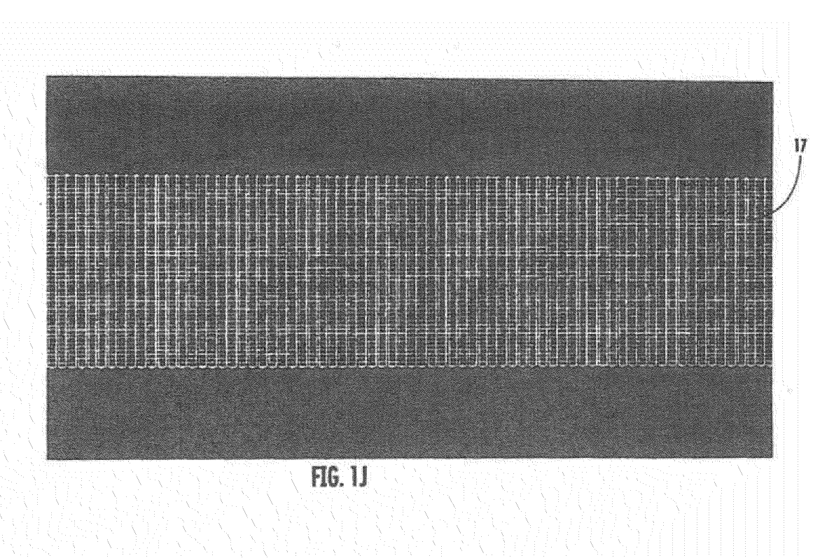

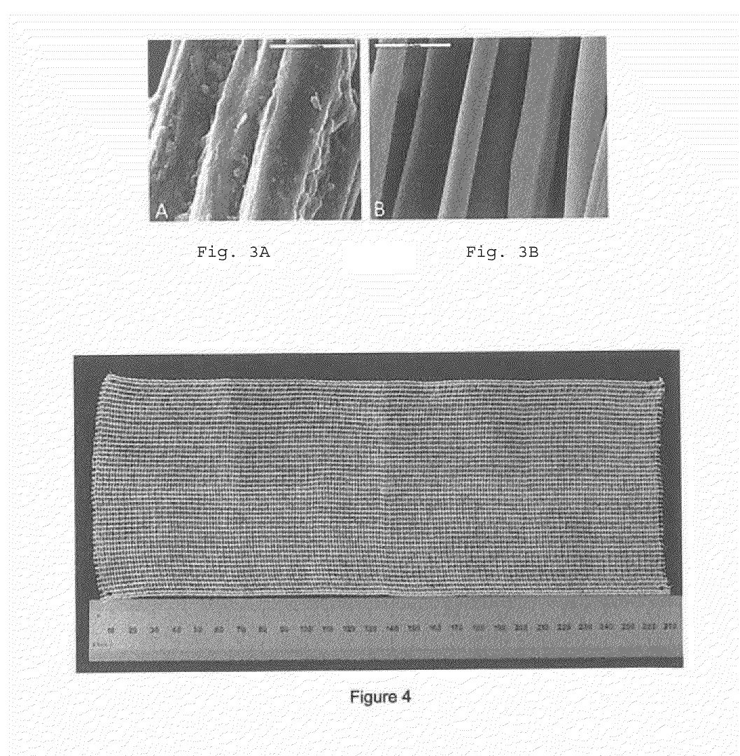

Importantly, the device made according to the present invention allows significant and consistent tissue ingrowth while bioresorbing at a rate which permits smooth transfer of load bearing support to the newly formed tissue. Thus the device is made of a biocompatible silk protein that is eventually bioresorbed. The raw silk fibers obtained from Bombyx mori silkworms comprise a fibroin protein core filament coated with the antigenic globular protein sericin. The sericin is removed or substantially all removed by hot aqueous (i.e. soap) extraction (wash) leaving behind fibroin protein filament consisting of layers of antiparallel beta sheets which provide both stiffness and toughness. FIG. 3A is a SEM photograph of native (sericin coated) silk fibers, and FIG. 3B of the fibers after sericin extraction, as then used to make (knit) the device. The porous knit structure of the device so made is shown by FIGS. 1A, 4 and 5.

Multiple sericin-depleted fibroin protein fibers are combined and twisted together to form a multi-filament yarn. The multi-filament fibroin yarn is subsequently knitted into a three dimensional pattern to serve as soft tissue support and repair. The resulting device is mechanically strong, flexible, and tear-resistant. The device is a single use only scaffold that can be produced in a variety of shapes, sizes and thicknesses and can be terminally sterilized.

The device provides immediate physical and mechanical stabilization of tissue defects because of its strength and porous construction and is useful as a transitory scaffold for soft tissue support and repair. It provides reinforcement for deficiencies where weakness or voids exist that require additional material reinforcement to obtain the desired surgical outcome. The bioresorption process occurs over time after implantation of the device as tissue in-growth and neovascularization takes place.

The device can be used to assist soft tissue repair. Examples of soft tissue repair include breast reconstruction, hernia repair, cosmetic surgery, implementation of a bladder sling, or the like.

Silk is the material used to make the device. Particular embodiments may be formed from Bombyx Mori silkworm silk fibroin. As explained a preferred embodiment of the device is made using sericin extracted silk fibers with certain knit machine parameters or settings. A detailed explanation of the knit pattern and knit process used to make a most preferred embodiment of the present invention will now be set forth. FIG. 1A is a photograph of a pattern layout for a device (silk-based mesh or scaffold) in accordance with the present invention. FIG. 1A shows the wale direction 10 and the course direction 15 and placement of the silk yarns in either the wale 10 or course 15 scaffold material direction or location. The device is preferably formed on a raschel knitting machine such as Comez DNB/EL-800-8B set up in 10 gg needle spacing by the use of three movements as shown in pattern layout in FIGS. 1B and 1C: two movements in the wale direction, the vertical direction within the fabric, and one movement in the course direction, the horizontal direction of the fabric. The movements in the wale direction occur on separate needle beds with alternate yarns; loops that occur on every course are staggered within repeat. The yarn follows a repeat pattern of 3/1-1/1-1/3-3/3 for one of the wale direction movements as shown in FIGS. 1D and 1E and 1/1-1/3-3/3-3/1 for the other wale direction movement as shown in FIGS. 1H and 1I. The interlacing of the loops within the fabric allows for one yarn to become under more tension than the other under stress, locking it around the less tensioned yarn, thereby keeping the fabric from unraveling when cut. The other movement in the course direction as shown in FIGS. 1F and 1G occurs in every few courses creating the porous design of the device. These yarns follow a repeat pattern of 7/7-9/9-7/7-9/9-7/7-9/9/-1/1-1/1-3/3-1/1-3/3-1/1-3/3-1/1-3/3-1/1 for the course direction movement. The pattern simulation layout of this pattern was rendered using ComezDraw 3 software in FIG. 1J considering a yarn design made with 3 ends of Td (denier count) 20/22 raw silk twisted together in the S direction to form a ply with 20 tpi (turns per inch) and further combining three of the resulting ply with 10 tpi. In FIG. 1J The same yarn design is used for the movements occurring in the wale and course directions. The stitch density or pick count for the design in FIG. 1J is 34 picks per centimeter considering the total picks count for the technical front face and the technical back face of the fabric, or 17 picks per cm considering only on the face of the fabric. The operating parameters described in FIGS. 1B to 1I are the optimum values for the specific yarn design used for the pattern simulation layout of FIG. 1J. In FIG. 1J item 17 is a simulated double needle bed mesh or scaffold. To further explain aspects shown by FIG. 1F: following standard terminology well known in the knit industry “F” means front and “B” means back and with regard to FIG. 1F shows the incremental sequence of pattern lines for the course direction. The numbers “12, 9, 6 and 3” at the bottom of FIG. 1F represent the number of needles in the needle bed starting count from left to right. The upwards pointing arrows near the bottom of FIG. 1F show the needle slots occupied by a needle actively engaged with the yarn for the knit machine/knit process. Rows 1F to 6B of FIG. 1F show the knit pattern used to make the device. The FIG. 1F rows 1F to 6B knit pattern is repeated 94 times to make a 25 cm sheet length of the fabric of the device, the number of repeats of the pattern being fewer or more if respectively a smaller or larger section of device fabric is desired to result from the knit process. The FIG. 1F rows 1F to 6B knit pattern is equivalently described by the above set forth, combined three knit movement: the first wale direction 3/1-1/1-1/3-3/3 knit pattern; the second wale direction 1/1-1/3-3/3-3/1 knit pattern, and; the course direction 7/7-9/9-7/7-9/9-7/7-9/9/-1/1-1/1-3/3-1/1-3/3-1/1 knit pattern. Rows 7F to 10B in FIG. 1F (and equivalently the terminal 3/3-1/1-3/3-1/1-3/3-1/1-3/3-1/1 portion of the course knit pattern) show the knit pattern used to make a spacer which creates a knitted area of fabric separation (i.e. a cut location) between adjacent 25 cm lengths of the knitted device fabric which is knitted by the process set forth above as one continuous sheet of fabric. The specific feed rates for the yarn forming this most preferred embodiment of the device is shown in FIG. 1 K where column 17 shows the yarn feed rate used for the first wale direction 3/1-1/1-1/3-3/3 knit pattern. A rate of 212 is equivalent to 74.8 cm of yarn per 480 coursed or per rack. Column 23 of FIG. 1K reports the yarn feed rate that is used for the second wale direction 1/1-1/3-3/3-3/1 knit pattern, where again a rate of 212 is equivalent to 74.8 cm of yarn per 480 coursed or per rack. Column 22 of FIG. 1K shows the yarn feed rate that is used for the course direction 7/7-9/9-7/7-9/9-7/7-9/9/-1/1-1/1-3/3-1/1-3/3-1/1-3/3-1/1-3/3-1/1-3/3-1/1-3/3-1/1 knit pattern; a rate from line 1F to 6B of 190 is equivalent to 67.0 cm of yarn per 480 coursed or per rack, while a rate from line 7F to 10B of 90 is equivalent to 31.7 cm of yarn per 480 coursed or per rack. Column 21 of FIG. 1K shows that the yarn feed rate that is used for the second to last yarn at each edge of the knitted device fabric in the course direction 7/7-9/9-7/7-9/9-7/7-9/9/-1/1-1/1-3/3-1/1-3/3-1/1-3/3-1/1-3/3-1/1-3/3-1/1-3/3-1/1 knit pattern; a rate from line 1F to 6B of 130 is equivalent to 45.8 cm of yarn per 480 coursed or per rack, while a rate from line 7F to 10B of 90 is equivalent to 31.7 cm of yarn per 480 coursed or per rack. Column 20 of FIG. 1K reports (shows) the yarn feed rate that is used for the last yarn at each edge of the knitted device fabric in the course direction 7/7-9/9-7/7-9/9-7/7-9/9/-1/1-1/1-3/3-1/1-3/3-1/1-3/3-1/1-3/3-1/1-3/3-1/1-3/3-1/1 knit pattern; a rate from line 1F to 6B of 130 is equivalent to 45.8 cm of yarn per 480 coursed or per rack, while a rate from line 7F to 10B of 90 is equivalent to 31.7 cm of yarn per 480 coursed or per rack.

The knit pattern shown in FIG. 1A can be knit to any width depending upon the knitting machine and can be knitted with any of the gauges available with the various crochet machines or warp knitting machines. Table 1 outlines the device fabric widths that may be achieved using a different numbers of needles on different gauge machines. The dimensions in Table 1 are approximate due to the shrink factor of the knitted fabric which depends on stitch design, stitch density, and yarn size used.

TABLE 1

| | |
|---|---|
| Needle | Knitting Width |
| Count | (mm) |

| Gauge | From | To | From | To |
|---|---|---|---|---|
| 48 | 2 | 5656 | 0.53 | 2997.68 |
| 24 | 2 | 2826 | 1.06 | 2995.56 |
| 20 | 2 | 2358 | 1.27 | 2994.66 |
| 18 | 2 | 2123 | 1.41 | 2993.43 |
| 16 | 2 | 1882 | 1.59 | 2992.38 |
| 14 | 2 | 1653 | 1.81 | 2991.93 |
| 12 | 2 | 1411 | 2.12 | 2991.32 |
| 10 | 2 | 1177 | 2.54 | 2989.58 |
| 5 | 2 | 586 | 5.08 | 2976.88 |

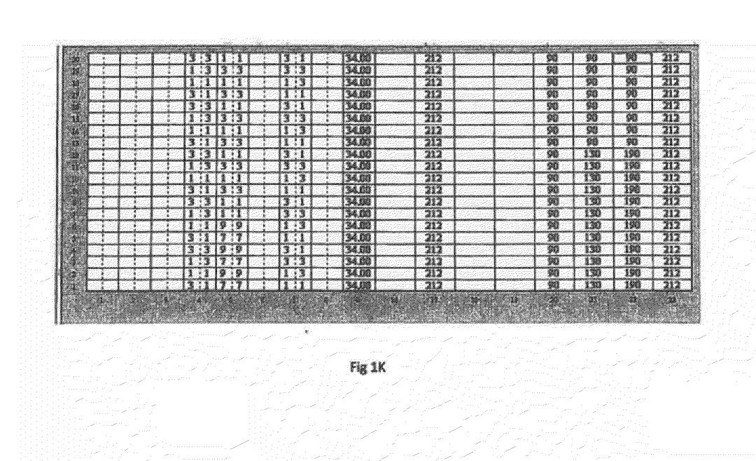

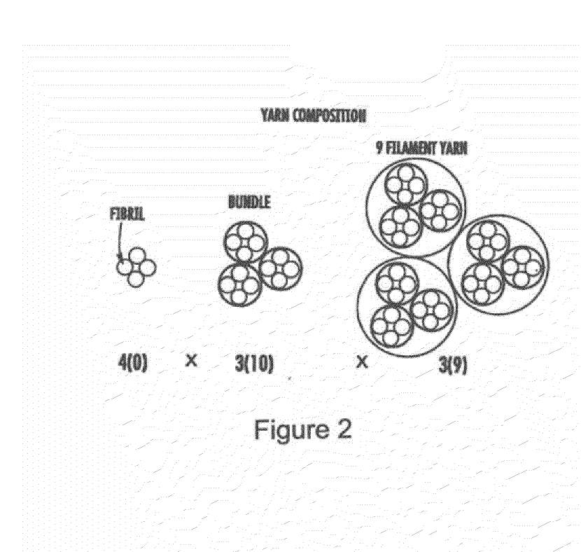

The device was knit with 9-filament, twisted silk yarns. A yarn was made from three silk bundles, each of which was comprised of individual silk fibrils as illustrated in FIG. 2. The 9-filament yarns were knit into the surgical scaffold. The wales ran horizontally and the courses ran vertically along the scaffold.

A preferred embodiment of the device ready for surgical use has a thickness between about 0.6 mm and about 1.0 mm, a width of about 10 cm (± about 1 cm) and a length of about 25 cm (± about 3 cm). Additionally the device has pores with an average diameter greater than about 10,000 um², a density of from about 0.14 mg/mm³ to about 0.18 mg/mm³ (as determined by dividing the mass of the device by its volume [thickness, width, and length multiplied together]), and is comprised of at least about 95% silk fibroin. Furthermore, the device has a burst strength of from about 0.54 MPa to about 1.27 MPa, and a stiffness of between about 30 N/mm to about 50 N·mm (the latter two mechanical properties of the preferred device determined by American Society for Testing and Materials D3787-07, “Standard Method for Burst Strength of Textiles: Constant Rate of Transverse Ball Burst Test” or ASTM F2150-07 Standard Guide for Characterization and Testing of Biomaterial Scaffolds Used in Tissue Engineered Medical Products)

The density of the device was calculated using the equation:

$$Material Density = \frac{Mass [ mg ]}{\begin{matrix}( Average Length [ mm ] ) \times ( Average Width [ mm ] ) \times \\ ( Thickness [ mm ] )\end{matrix}}$$

The cross-sectional area of full pores of the scaffold was measured using a microscope with sufficient magnification and image capture capability. The magnification was selected based upon the resolution of the pores in the knit pattern being examined.

Ball Burst Testing—

Per ASTM D3787-07, each device tested was compressed between the two circular fixation brackets of the mechanical testing equipment, while leaving exposed a circular area of the test article that covers the radius of the inner fixture diameter. The sample device was secured with a constant fixation bolt torque to the locking nuts of the burst jig. Care was taken to ensure that the knit structure of the sample was organized and not skewed or sheared. The sample remained taut within the fixation brackets with equal distribution of tension. The ball burst fixture was attached to the mechanical testing equipment with a calibrated load cell. For the burst test, the fixture ball was inserted through the center diameter of the fixation brackets with a uniform pressure applied to the test article. The ball was inserted at a constant rate until the scaffold fails.

Burst stiffness was calculated by determining the slope of the middle 60% of the linear region of the compressive load vs. extension curve.

Maximum burst strength was calculated using the equation:

$$Maximum Burst Strength [ MPa ] = [ \frac{Maximum Burst Load [ N ]}{Exposed Area [ {m}^{2} ]} ] \times {10}^{- 6}$$

The exposed area was the circular area of the test article covering the radius (r) of the inner fixture diameter and was calculated using the equation below.

Exposed Area=πr²

Tensile Testing—

The tensile strength and elongation of the device were measured in accordance with ASTM D5035. Device samples were clamped in the mechanical test equipment. The upper clamp was mounted to the load cell, which was attached to the actuator and the lower clamp was mounted to the support plate. The lower limit of the actuator was set so that the upper and lower clamps were prevented from colliding. The upper clamp was aligned to make the faces of both clamps parallel to each other. The height of the mechanical equipment crosshead was adjusted so that the actuator was positioned to allow for a defined amount of upward movement and a specific sample gauge length resided between the upper and lower sample clamps.

The device was loaded by clamping the first 10 mm of the sample into the upper clamp and allowing the remainder of the sample to fall unrestrained into the bottom clamp opening. The last 10 mm of the sample was held by the bottom clamp. Care was taken to avoid pre-staining the device sample. Once the sample was clamped the actuator height was adjusted so that the sample had a pre-load of 2 N. The actuator position was adjusted to achieve a specific gauge length and then reset to the zero-position at this point. The device sample was strained until it experienced ultimate tensile failure. The average maximum tensile strength, maximum tensile stress, percent elongation at break, and the tensile stiffness were determined. Tensile stiffness was calculated by determining the slope of the trend line of the linear portion of the tensile load vs. elongation curve bound by an upper and lower tensile load.

Tensile stiffness was calculated as the slope of the linear portion of the load verses elongation curve. The average maximum tensile strength, maximum tensile stress, linear stiffness, and percent elongation at break were determined.

Maximum tensile stress was calculated using the equation:

$$Maximum Tensile Stress [ MPa ] = [ \frac{Maximum Tensile Strength [ N ]}{Width [ m ] \times Thickness [ m ]} ] \times {10}^{- 6}$$

Whereby, the thickness and width were provided by the respective device sample thickness and width measurements.

Percent elongation at break was determined using the equation:

$$PercentElongationatBreak [ \% ] = [ \frac{Elongation at Break [ mm ]}{Length [ mm ]} ] \%$$

Whereby, length was provided by the respective device sample length measurement.

Tear Testing—

A device sample with a width that is two-thirds that of the length was cut from each device. Before the samples are incubated in phosphate buffered saline, a small cut that was one-fourth the size of the sample width was made in the center of the device sample perpendicular to the length (through a single row of wales). Mechanical test equipment was used to measure the maximum tear resistance load. Clamps were inserted in the equipment. The upper clamp was mounted to the load cell that was attached to the actuator and the lower clamp was mounted to the base support plate. The lower limit of the actuator was set so that the upper and lower clamps were prevented from colliding. The upper clamp was aligned to make the faces of both clamps parallel to each other. The height of the mechanical equipment crosshead was adjusted so that the actuator was positioned to allow for a defined amount of upward movement and a specific sample gauge length resided between the upper and lower clamps. The device sample was placed in the upper clamp. The top 10 mm of the sample was covered by the clamp. The device sample was positioned so that the cut was located on the left side. The sample was aligned perpendicular with the clamp before the clamp was closed. The bottom portion of the sample was allowed to fall unrestrained into the bottom clamp opening. The clamp was closed and the sample was preloaded with 3 N. The sample was strained at a constant rate until the sample tore at the cut point. From the resulting data the maximum tear resistance load was obtained.

Embodiments of the device according to the present invention can be knitted on a fine gauge crochet knitting machine. A non-limiting list of crochet machines capable of manufacturing the surgical mesh according to aspects of the present invention are provided by: Changde Textile Machinery Co., Ltd.; Comez; China Textile Machinery Co., Ltd.; Huibang Machine; Jakkob Muller AG; Jingwei Textile Machinery Co., Ltd.; Zhejiang Jingyi Textile Machinery Co., Ltd.; Dongguan Kyang the Delicate Machine Co., Ltd.; Karl Mayer; Sanfang Machine; Sino Techfull; Suzhou Huilong Textile Machinary Co., Ltd.; Taiwan Giu Chun Ind. Co., Ltd.; Zhangjiagang Victor Textile; Liba; Lucas; Muller Frick; and Texma.

Embodiments of the device according to the present invention can be knitted on a fine gauge warp knitting machine. A non-limiting list of warp knitting machines capable of manufacturing the surgical mesh according to aspects of the present invention are provided by: Comez; Diba; Jingwei Textile Machinery; Liba; Lucas; Karl Mayer; Muller Frick; Runyuan Warp Knitting; Taiwan Giu Chun Ind.; Fujian Xingang Textile Machinery; and Yuejian Group.

Embodiments of the device according to the present invention can be knitted on a fine gauge flat bed knitting machine. A non-limiting list of flat bed machines capable of manufacturing the surgical mesh according to aspects of the present invention are provided by: Around Star; Boosan; Cixing Textile Machine; Fengshen; Flying Tiger Machinary; Fujian Hongqi; G & P; Görteks; Jinlong; JP; Jy Leh; Kauo Heng Co., Ltd.; Matsuya; Nan Sing Machinery Limited; Nantong Sansi Instrument; Shima Seiki; Nantong Tianyuan; and Ningbo Yuren Knitting.

A test method was developed to check the cutability of the device formed according to aspects of the present invention. In the test method the device evaluated according to the number of scissor strokes needed to cut the device with surgical scissors. The mesh was found to cut excellently because it took only one scissor stroke to cut through it. The device was also cut diagonally and in circular patterns determining that the device did not unraveled once cut in either or both its length and width directions (see FIG. 5B). To determine further if the device would unravel a suture was passed through the closest pore from the cut edge, and pulled. This manipulation did not unravel the device. Thus the device was easy to cut and did not unravel after manipulation.

A device according to the present invention has been found to bioresorb by 50% in approximately 100 days after implantation, that is at least about 50% of the mass of the device bioresorbs after about 100 days after implantation in a human patient.

Physical properties of the device include thickness, density and pore sizes. The thickness of the device was measured utilizing a J100 Kafer Dial Thickness Gauge. A Mitutoyo Digimatic Caliper was used to find the length and width of the samples; used to calculate the density of the device. The density was found by multiplying the length, width and thickness of the mesh then dividing the resulting value by the mass. The pore size of the device was found by photographing the mesh with an Olympus SZX7 Dissection Microscope under 0.8× magnification. The measurements were taken using ImagePro 5.1 software and the values were averaged over several measurements. Physical characteristics of sample meshes, and two embodiments of the device are shown in Table 2.

TABLE 2

| | Physical Characterization | | |
|---|---|---|---|
| | Thickness | | |
| Sample | (mm) | Pore Size (mm²) | Density (g/cm³) |
| Mersilene Mesh | 0.31 ± 0.01 | 0.506 ± 0.035 | 0.143 ± 0.003 |
| Bard Mesh | 0.72 ± 0.00 | 0.465 ± 0.029 | 0.130 ± 0.005 |
| Vicryl Knitted Mesh | 0.22 ± 0.01 | 0.064 ± 0.017 | 0.253 ± 0.014 |
| Device knit on a | 1.00 ± 0.04 | 0.640 ± 0.409 | 0.176 ± 0.002 |
| single needle bed | | | |
| machine | | | |
| Device knit on a | 0.89 ± 0.003 | 1.26 ± 0.400 | 0.165 ± 0.005 |
| double needle bed | | | |
| machine | | | |

To summarize a device according to the present invention is a biocompatible, bioresorbable, surgical matrix (mesh or scaffold) made preferably from the silk of the Bombyx mori silkworm. Because raw silk fibers are comprised of a fibroin protein core filament that is naturally coated with the antigenic globular protein sericin the sericin is removed by aqueous extraction. Yarn is then made from the sericin-depleted fibroin protein filaments by helical twisting to form a multi-filament protein fiber. The multi-filament protein fiber yarn is then knit into a three dimensional patterned matrix (mesh or scaffold) that can be used for soft tissue support and repair. The device upon implantation provides immediate physical and mechanical stabilization of tissue defects because of its strength and porous construction. Additionally, the porous lattice design of the device facilitate native tissue generation (that is tissue ingrowth) and neovascularization. The natural tissue repair process begins with deposition of a collagen network. This network integrates within the protein matrix, interweaving with the porous construct. Neovascularization begins with endothelial cell migration and blood vessel formation in the developing functional tissue network. This new functional tissue network and its corresponding vascular bed ensure the structural integrity and strength of the tissue. In the beginning stages of the tissue ingrowth process, the device provides the majority of structural support. The device (made of silk) is gradually deconstructed (bioresorbed) into its amino acid building blocks. The slow progression of the natural biological process of bioresorption allows for the gradual transition of support from the protein matrix of the device to the healthy native tissue thereby achieving the desired surgical outcome.

What is claimed is:

1. A process for making a knitted silk mesh, the process comprising the steps of:
(a) knitting a first silk yarn in a first wale direction using the pattern 3/1-1/1-1/3-3/3;
(b) knitting a second silk yarn in a second wale direction using the pattern 1/1-1/3-3/3-3/1, and;
(c) knitted a third silk yarn in a course direction using the pattern 7/7-9/9-7/7-9/9-7/7-9/9/-1/1-1/1-3/3-1/1-3/3-1/1,
thereby obtaining a knitted ilk mesh.

2. The process of claim 1, wherein the knitting of the first silk yarn is carried out on a first needle bed and the knitting of the second silk yarn is carried out on a second needle bed.

3. The process of claim 1 wherein each of the three silk yarns is made with three ends of Td (denier count) 20/22 silk twisted together in the S direction to form a ply with 20 tpi (turns per inch) and further combining three of the resulting ply with 10 tpi.

4. The process of claim 1, where the knitted silk mesh has a stitch density or pick count of 34 picks per centimeter with regard to the total picks count for the technical front face and the technical back face of the knitted silk mesh.

5. A process for making a knitted silk mesh, the process comprising the steps of:
(a) knitting a first silk yarn in a first wale direction using the pattern 3/1-1/1-1/3-3/3;
(b) knitting a second silk yarn in a second wale direction using the pattern 1/1-1/3-3/3-3/1, and;
(c) knitted a third silk yarn in a course direction using the pattern 7/7-9/9-7/7-9/9-7/7-9/9/-1/1-1/1-3/3-1/1-3/3-1/1.
wherein:
(d) wherein the knitting of the first silk yarn is carried out on a first needle bed and the knitting of the second silk yarn is carried out on a second needle bed;
(e) each of the three silk yarns is made with 3 ends of Td (denier count) 20/22 raw silk twisted together in the S direction to form a ply with 20 tpi (turns per inch) and further combining three of the resulting ply with 10 tpi, and;
(f) the knitted silk mesh has a stitch density or pick count of 34 picks per centimeter with regard to the total picks count for the technical front face and the technical back face of the knitted silk mesh.

6. A knitted silk mesh made by:
(a) knitting a first silk yarn in a first wale direction using the pattern 3/1-1/1-1/3-3/3;
(b) knitting a second silk yarn in a second wale direction using the pattern 1/1-1/3-3/3-3/1, and;
(c) knitted a third silk yarn in a course direction using the pattern 7/7-9/9-7/7-9/9-7/7-9/9/-1/1-1/1-3/3-1/1-3/3-1/1.

7. The knitted silk mesh of claim 6, wherein the knitting of the first silk yarn is carried out on a first needle bed and the knitting of the second silk yarn is carried out on a second needle bed.

8. The knitted silk mesh of claim 6, wherein each of the three silk yarns is made with 3 ends of Td (denier count) 20/22 raw silk twisted together in the S direction to form a ply with 20 tpi (turns per inch) and further combining three of the resulting ply with 10 tpi;
thereby making a knitted silk mesh with a stitch density or pick count of 34 picks per centimeter with regard to the total picks count for the technical front face and the technical back face of the knitted silk mesh.

9. A knitted silk mesh made by:
(a) knitting a first silk yarn in a first wale direction using the pattern 3/1-1/1-1/3-3/3;
(b) knitting a second silk yarn in a second wale direction using the pattern 1/1-1/3-3/3-3/1, and;
(c) knitted a third silk yarn in a course direction using the pattern 7/7-9/9-7/7-9/9-7/7-9/9/-1/1-1/1-3/3-1/1-3/3-1/1;
wherein:
(d) the knitting of the first silk yarn is carried out on a first needle bed and the knitting of the second silk yarn is carried out on a second needle bed, and;
(e) each of the three silk yarns is made with 3 ends of Td (denier count) 20/22 raw silk twisted together in the S direction to form a ply with 20 tpi (turns per inch) and further combining three of the resulting ply with 10 tpi;
thereby making a knitted silk mesh with a stitch density or pick count of 34 picks per centimeter with regard to the total picks count for the technical front face and the technical back face of the knitted silk mesh.

10. A knitted silk mesh comprising:
(a) a thickness between about 0.6 mm and about 1.0 mm;
(b) pores with an average diameter greater than about 10,000 um², and;
(c) a density of from about 0.14 mg/mm³ to about 0.18 mg/mm³.

11. The knitted silk mesh of claim 10, further comprising a burst strength of from about 0.54 MPa to about 1.27 MPa.

12. The knitted silk mesh of claim 10 further comprising a stiffness of between about 30 N/mm to about 50 N·mm.

13. The knitted silk mesh of claim 10, wherein at least about 50% of the knitted silk mesh has bioresorbed after about 100 days after implantation in a human patient.

14. A knitted silk mesh comprising:
(a) a thickness between about 0.6 mm and about 1.0 mm;
(b) pores with an average diameter greater than about 10,000 um²;
(c) a density of from about 0.14 mg/mm³ to about 0.18 mg/mm³;
(d) a burst strength of from about 0.54 MPa to about 1.27 MPa;
(e) a stiffness of between about 30 N/mm to about 50 N/mm, and
(f) wherein at least about 50% of the knitted silk mesh has bioresorbed after about 100 days after implantation in a human patient.
